(19) United States
(12) Patent Application Publication
(10) Pub. No.: US 2004/0014788 A1
(43) Pub. Date: Jan. 22, 2004

(54) Novel uses of combined selective dopamine d2 receptor antagonists and 5-ht receptor agonists

(76) Inventor: Gerd Bartoszyk, Weiterstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/416,575

(22) PCT Filed: Oct. 25, 2001
(86) PCT No.: PCT/EP01/12325

(30) Foreign Application Priority Data
Nov. 14, 2000 (EP) 00124814.5

(51) Int. Cl. A61K 31/4433; A61K 31/277

(52) U.S. Cl. 514/337; 514/524

(57) ABSTRACT

Use of compounds being combined selective dopamine D2 receptor antagonists and 5-HT_{1A} receptor agonists, in particular (R)-(−)-2-[5-4 fluorophenyl)-3-pyridylmethyl-aminomethyl]-chromane or a physiologically acceptable salt thereof or N-(4′-fluoro-3-biphenylmethyl)-N-2-(3-cycanophenoxy-ethyl)-amine or a physiologically acceptable salt thereof, for the manufacture of a medicament for use in veterinary medicine for the treatment of self directed traumatic disorders associated with behavioral stressors, compulsive disorders associated with behavioral stressors and/or anxiety disorders associated with behavioral stressors.

The present invention relates to the use of compounds being combined selective dopamine D2 receptor antagonists and 5-HT_{1A} receptor agonists for the manufacture of a medicament for use in veterinary medicine for the treatment or prophylaxis of disorders associated with behavioral stressors

Particularly, the present invention relates to the use of combined selective dopamine D2 receptor antagonists and 5-HT_{1A} receptor agonists chosen from the group consisting of (R)-(−)-2-[5-(4-fluorophenyl)-3-pyridylmethylaminomethyl]-chromane or physiologically acceptable salts thereof or N-(4′-fluoro-3-biphenylmethyl)-N-2-(3-cyano-phenoxy-ethyl)-amine or a physiologically acceptable salt thereof, for the manufacture of a medicament for use in veterinary medicine for the treatment of self directed traumatic disorders associated with behavioral stressors and/or compulsive disorders associated with behavioral stressors and/or anxiety disorders associated with behavioral stressors.

(R)-(−)-2-[5-(4-fluorophenyl)-3-pyridylmethylaminomethyl]-chromane, physiologically acceptable salts thereof (U.S. Pat. No. 5,767,132, column 9, lines 6 to 32) and a process (U.S. Pat. No. 5,767,132, Example 19) by which it/they can be prepared are known from U.S. Pat. No. 5,767,132. The compound which is referred to herein is described in the patent as a combined selective dopamine D2 receptor antagonist and 5-HT_{1A} receptor agonist. N-(4′-fluoro-3-biphenylmethyl)-N-2-(3-cyano-phenoxy-ethyl)-amine, physiologically acceptable salts thereof (U.S. Pat. No. 5,767,132, column 9, lines 6 to 32) and a process (U.S. Pat. No. 5,767,132, Example 6) by which it/they can be prepared are known from U.S. Pat. No. 5,767,132. The compound which is referred to herein is described in the patent as a combined selective dopamine D2 receptor antagonist and 5-HT_{1A} receptor agonist.

Therefore, the use of (R)-(−)-2-[5-(4-fluorophenyl)-3-pyridylmethylaminomethyl]-chromane and its physiologically acceptable acid addition salts and the use of N-(4′-fluoro-3-biphenylmethyl)-N-2-(3-cyano-phenoxy-ethyl)-amine and its physiologically acceptable salts thereof for the manufacture of a medicament for prophylaxis and control of the sequelae of cerebral infarction (apoplexia cerebri) such as stroke and cerebral ischaemia, for prophylaxis and control of cerebral disorders, e.g. migraine, especially in geriatrics in a manner similar to certain ergot alkaloids, the treatment of anxiety, tension and depression states, sexual dysfunctions caused by the central nervours system, for disturbances in sleep or absorption of food or for the treatment of psychosis (schizophrenia) is disclosed.

Additionally, they are suitable to eliminate cognitive deficiencies, to improve powers of learning and memory and to treat Alzheimer's disease. They can be furthermore used for treating side-effects in the treatment of hypertension, in endocrinology and gynecology, e.g. for the treatment of acromegaly, hypogonadism, secondary amenorrhea, premenstrual syndrome or undesired puerperal lactation.

The invention had the object of providing novel uses for compounds being combined selective dopamine D2 receptor antagonists and 5-HT_{1A} receptor agonists, in particular for (R)-(−)-2-[5-(4-fluorophenyl)-3-pyridylmethylaminomethyl]-chromane and its physiologically acceptable salts or N-(4′-fluoro-3-biphenylmethyl)-N-2-(3-cyano-phenoxy-ethyl)-amine or a physiologically acceptable salt thereof, especially in the field of veterinary medicine.

It has been found that compounds being combined selective dopamine D2 receptor antagonists and 5-HT_{1A} receptor agonists, in particular (R)-(−)-2-[5-(4-fluorophenyl)-3-pyridylmethylaminomethyl]-chromane or its physiologically acceptable salts or N-(4′-fluoro-3-biphenylmethyl)-N-2-(3-cyano-phenoxy-ethyl)-amine or a physiologically acceptable salt thereof, also have activity against disorders associated with behavioral stressors in veterinary medicine.

Disorders associated with behavioral stressors include self directed traumatic disorders (also known as self-injurious behavior), compulsive disorders and anxiety disorders which all are associated with behavioral stressors (e.g. C. C. Pinney, The illustrated veterenary guide for dogs, cats, birds and exotic pets. McGraw Hill, 1992; U. A. Luescher, in: N. H. Dodman and L. Shuster (eds.): Psychopharmacology of Animal Behavior, Blackwell Science Inc., Maiden, 1998, pp. 203-221).

Self directed traumatic disorders associated with behavioral stressors and compulsive disorders associated with behavioral stressors result particularly in stereotyped behaviors in animals, a common problem for pet owners, breeders, zoo keepers and veterinarians. Self directed traumatic disorders are characterized by self-injurious behaviors such as acral lick dermatitis (ALD), flank sucking, spinning and tail chaising, nail biting and checking of the rear end in dogs, psychogenic alopecia (hair pulling), tail attacking, pawing the face and hyperesthesia in cats, or feather picking (trichotillomania) in birds.

Compulsive disorders are seen in other species, too, e.g. repetitive or ritualized pacing seen in zoo animals, cribbing, self-mutation or weaving in horses.

For example, intense animal keeping is characterized by high animal populations on restricted grounds. Animals kept under such conditions experience extreme stress and develop both traumatic and compulsive disorders, e.g. bar biting in pigs in meat-producing farms or feather picking in hens in egg-producing farms (e.g. M. Kiley-Worthinton, Behavioral problems of farm animals, Orial Press, Stockfields, 1977; R. J. Young et al., Appl. Anim. Behav. Sci., 1994,39: 237-247).

Anxiety disorders are thought to be comorbid with self directed traumatic disorders and some compulsive disorders because of the identical circumstances when they develop.

Amongst these disorders, in veterinary practice especially the self directed traumatic disorders, i.e. acral lick dermatitis in dogs, psychogenic alopecia in cats, feather picking in caged birds, and self-mutilation, cribbing and weaving in horses, are very common and usually the reason why pet owners consult a veterinarian (e.g. R. R. Keiper, Anim. Behav., 1970, 18: 353-357; G. G. Vecciotti and R. Galanti, Livestock Prod. Sci., 1986, 14: 9195; C. Davis, Vet. Clin. N Am. Sm. An. Pract., 1991, 21: 1281-1288; C. C. Pinney, The illustrated veterenary guide for dogs, cats, birds and exotic pets. McGraw Hill, 1992; D. J. Stein and N. H. Dodman, Comp. Psychiatr., 1994, 35: 275-285).

Acral lick dermatitis is a condition characterized by excessive paw licking and scratching in dogs. This results in the characteristic dermatitis; other sequelae include acute and chronic osteomyelitis. Acral lick dermatitis, also known as lick granuloma, creates areas of hair loss and the production of lesions, which may range in size from several centimeters to the entire surface of the limb, finally inflamed and ulcerated causing discomfort, pain, and in severe cases crippling. Osteomyelitis represents a suppurative infection of bone tissue with a progressive course with signs of bone destruction and formation of atrophic foci in the bone; in some cases sequestra and false joints are formed. The disorder is seen in certain breeds of large dog (particularly prone are Doberman Pinscher, German Shephard, Great Danes, Golden Retriever, Labrador Retriever, Irish Setter, and Weimaraner), and may be more common in particular families within breeds (e.g. L. Veith, Canine Pract., 1986, 14: 15-22; J. J. Van Nes, J. Am. Vet. Med. Assoc., 1986,198:157-160; S. D. White, J. Am. Vet. Med. Assoc., 1990, 202: 1073-1076; B. A. Goldberger and J. L. Rapoport, J. Anim. Hosp. Assoc., 1990, 27: 179-182; U. A. Luescher et al., Vet. Clin. N Am. Sm. Anim. Pract., 1991, 21: 401-413; K. Overall, J. Am. Vet. Assoc., 1994, 205: 1733-1741; U. A. Luescher, Vet. Intern., 1998, 10: 7-12).

Psychogenic alopecia is found in cats, where excessive depilation leads to bare patches (e.g. U. A. Luescher et al., Vet. Clin. N Am. Sm. Anim. Pract., 1991, 21: 401-413; J. W. S. Bradshaw et al., J. Appl. An. Behav. Sci., 1997, 52: 373-379; J. Dehasse, Appl. An. Behav., 1997, 52: 365-371; L. S. Sawyer et al., J. Am. Vet. Med. Assoc., 1999, 214: 71-74). Animal behaviorists have typically viewed the disorder as stress related. All strains are prone of psychogenic alopecia.

Feather picking in birds is seen in a range of avian species. Complications associated with this disorder include severe haemorrhage, infection, and hypothermia. It is also known that stress and confinement play a role in this behavior. Very prone are African greys and Timneh greys, budgerigars, rosellas, neophemas and other Australian parakeets, cockatoos, conures, electus parrots, lorikeets, lovebirds, and macaws (e.g. R. R. Keiper, Anim. Behav., 1970, 18: 353-357; D. Alderton, An essential reference for keeping more than 200 parrot family species, Salamander Books, 1992; G. A. Gallerstein, The complete bird owner's handbook, Macmillan, 1994; C. Davis, Vet. Clin. N Am. Sm. An. Pract., 1991, 21:1281-1288; F. Iglauer and R. Rasim, J. Sm. An. Pract., 1993, 34: 564-566; P. S: Brodnick et al., J. Behav. Ther. Exp. Psychiatr., 1994, 25: 189-196; S. Blanchard, Pet Bird Report, 1995, 23; S. V. Juarbe-Diaz, J. Am. Vet. Med. Assoc., 2000,1562-1564).

Self-mutilation behavior in horses consists of self-biting (flanks, limbs, lateral thoracic wall, pectoral area, tail) and other uncontrollable violent behaviors typically including spinning in circles, bucking, rubbing, kicking out with hind limbs sometimes while nipping at the flanck, shoulders or chest, and vocalization. In extreme cases, the horse can violently lunge its body or head into a wall or other solid suject. A single episode can last to several minutes uninterrupted, and episodes can be repeated for hours over a day. In addition to bite wounds, the most common injuries are to the legs and feet from spinning and kocking. Self-mutilation is most common in Arabian, Quarterhorse and American Standardbred stallions, although it occurs in both sexes and in a variety of other breeds (e.g. A. F. Frazer, The behavior of the horse, CAB International, Oxon, 1992; L. C: Winskill et al., Appi. An Behav Sci, 1996, 48: 25-25).

Cribbing behavior is the most well known stable vice. Horses bite an object, flexes its neck, pulls back with its teeth, and swallows air. Cribbing not only damages the house surroundings, but could threaten its life. Cribbing leads to weight loss, poor performance, gaseous colic, and excessive tooth wear (e.g. N. H. Dodman et al., Am. J. Vet. Res., 1987, 48: 311-319; M. Minero et al., Proc. Measuring Behav, 1996).

Weaving behavior in horses is an odd behavior in which the horse rocks from foreleg to foreleg for long amounts of time. Weaving generally occurs in horses which are kept in stalls (e.g. A. F. Frazer, The behavior of the horse, CAB International, Oxon, 1992).

Treatment of these disorders is extremely difficult and often resistant to any treatment.

Clomipramine hydrochloride, a tricyclic antidepressant drug with serotonin (5-HT) reuptake inhibiting properties, is used for the treatment of self directed traumatic disorders or compulsive disorders or anxiety disorders (e.g. M. H. Grindlinger and E. Ramay, Proc. Assoc. Avian Vet., 1992; P. A. Mertens and N. H. Dodman, Kleintierpraxis, 1996, 41: 313-392; R. A. Eckstein and B. L. Hart, J. Am. An. Hosp. Assoc., 1996, 32: 225-230; R. A. Casey, Vet. Rec., 1998, 142: 587-588; K. Seksel and M. J. Lindeman, Aust. Vet. J., 1998, 76: 317-321; A. L. Podberscek et al., Vet. Rec., 1999, 145: 365-369; L. S: Saxyer et al., J. Am. Vet. Med. Assoc., 1999, 214: 71-74). Alternatively, selective serotonin (5-HT) reuptake inhibitors (SSRIs) such as fluoxetine are used in such species including horses (e.g. K. L. Overall, Can. Pract., 1996, 21: 20-24; P. A. Mertens, ESVCE Newsletter, 1997, 3: n^{o} ⅘; H. G. Nurnberg et al., Biol. Psychiatr., 1997, 41: 226-229; D. Wynchank and M. Berk, Depress. Anxiety, 1998, 8: 21-23; J. Romatowski, Feline Pract., 1998, 26:14-15).

Likewise, dopamine D2 blockers such as haloperidol have been reported to be effective in dogs, cats, birds, rabbits, horses, pigs and other species, including zoo animals and wild life animals (e.g. J. M. Hofineyr, J. S. Afr. Vet. Assoc., 1981, 52: 273-282; R. J. Danzer, Anim. Sci., 1986, 62:1776-1786; D. Kennes et al., Eur. J. Pharmacol., 1988,153:19-24; G. C. Gandini et al., J. S. Afr. Vet. Assoc., 1989, 60: 206-207; E. Von Borell and F. J.; Smith, Life Sci., 1991, 49: 309-314; F. Iglauer and R. Rasim, J. Sm. An. Pract., 1993, 34: 564-566; T. Willemse, Eur. Neuropsychopharmacol., 1994, 4: 39-45; F. Iglauer et al., Lab. Anim., 1995, 29: 385-293; H. G. Nurnberg et al., Biol. Psychiatr., 1997, 41: 226-229; U. A. Luescher, in: N. H. Dodman and L. Shuster (eds.): Psychopharmacology of Animal Behavior, Blackwell Science Inc., Maiden, 1998, pp. 203-221, Y. Uchida et al., J. Am. Vet. Med. Assoc., 1998, 212: 354-355).

Although serotonin 5-HT_{1A} receptor agonists are not established as a pharmacological treatment in animals, the 5-HT_{1A} receptor agonist busprione has been proposed at least for dogs and cats (B. L. Hart et al., J. Am. Vet. Med. Assoc., 1993, 203: 254-258; K. L. Overall, J. Am. Vet. Med. Assoc., 1994, 205: 694-696; W. Jochle, Tier\overline{a}rztl. Prax., 1998, 26: 410-421). But is long known that 5-HT_{1A} receptor agonists reduce stress and relieve anxiety in animals (e.g. J. E. Barrett and K. E. Vanover, Psychopharmacology, 1993,112:1-12; G. Griebel, Pharmac. Ther., 1995, 65: 319-395; F. G. Graeff et al., Pharmacol. Biochem. Behav., 1996, 54: 129-141) and man (e.g. J. P. Feighner et al., J. Clin. Psychiatr., 1982, 43: 103-108; A. F. Jacobson et al., Pharmacotherapy, 1985, 5: 290-296; J. J. Sramek et al., Depress. Anxiety, 1999, 9: 131-134). Moreover, 5-HT_{1A} receptor agonists have been shown be effective alone (J. P. Apter and L. A. Allen, J. Clin. Psychopharmacol., 1999, 19: 86-93) and to augment the effect of SSRIs (P. J. Markovitz et al., Am. J. Psychiatr., 1990, 147: 798-800) in obsessive-compulsive disorders in humans which resemble in various aspects (e.g. trichotillomania, onychophagia, excessive washing, obsessive checking) self directed traumatic disorders and compulsive disorders in animals (e.g. E. Yadin et al., Pharmacol. Biochem. Behav., 1991, 40: 311-315; J. L. Rapoport et al., Arch. Gen. Psychiatr., 1992, 49: 517-521; P. S. Bordnick et al., J. Behav. Ther. Exp. Psychiatr., 1994, 25: 189-196; D. J. Stein and N. H. Dodman, Comp. Psychiatr., 1994, 35: 275-285; H. G. Nurnberg et al., Biol. Psychiatr., 1997, 41: 226-229; H. Szechtman et al., Behav. Neurosci., 1998, 112: 1475-1485, Pol. J. Pharmacol., 1999, 51: 55-61).

Therefore, the combination of a dopamine D2 blocker and a 5-HT1A receptor agonist as realized in (R)-(−)-2-[5-(4-fluorophenyl)-3-pyridylmethylaminomethyl]-chromane or its physiologically acceptable salts represents an advantage over the use of a D2 blocker alone, especially because of the additional anxiolytic/stress-reducing properties with respect to the 5-HT_{1A} agonistic activity.

Accordingly, the present invention relates to the use of compounds being combined selective dopamine D2 receptor antagonists and 5-HT_{1A} receptor agonists, in particular of (R)-(−)-2-[5-(4-fluorophenyl)-3-pyridylmethylaminomethyl]-chromane or a physiologically acceptable salt thereof or N-(4′-fluoro-3-biphenylmethyl)-N-2-(3-cyano-phenoxy-ethyl)amine or a physiologically acceptable salt thereof, for the manufacture of a medicament for use in veterinary medicine for the treatment of self directed traumatic disorders including acral lick dermatitis in dogs, psychogenic alopecia in cats and feather picking in birds, and/or compulsive disorders and or anxiety disorders which are associated with behavioral stressors.

Accordingly, the present invention relates particularly preferred to the use of (R)-(−)-2-[5-(4-fluorophenyl)-3-pyridylmethylaminomethyl]-chromane or a physiologically acceptable salt thereof, for the manufacture of a medicament for use in veterinary medicine for the treatment of disorders associated with behavioral stressors.

The present invention relates also to the use of N-(4′-fluoro-3-biphenylmethyl)-N-2-(3-cyano-phenoxy-ethyl)-amine or a physiologically acceptable salt thereof, for the manufacture of a medicament for use in veterinary medicine for the treatment of disorders associated with behavioral stressors.

A preferred salt of (R)-(−)-2-[5-(4-fluorophenyl)-3-pyridylmethylaminomethyl]-chromane is (R)-(−)-2-[5-(4-fluorophenyl)-3-pyridylmethylaminomethyl]-chromane hydrochloride.

Therefore, the invention relates to the use for the manufacture of a medicament for use in veterinary medicine for the treatment of disorders associated with behavioral stressors in which the pharmacologically acceptable salt is (R)-(−)-2-[5-(4-fluorophenyl)-3-pyridylmethylaminomethyl]-chromane hydrochloride.

A preferred salt of N-(4′-fluoro-3-biphenylmethyl)-N-2-(3-cyano-phenoxyethyl)-amine is N-(4′-fluoro-3-biphenylmethyl)-N-2-(3-cyano-phenoxy-ethyl)amine maleate.

Therefore the invention relates to the use for the manufacture of a medicament for use in veterinary medicine for the treatment of disorders associated with behavioral stressors in which the pharmacologically acceptable salt of N-(4′-fluoro-3-biphenylmethyl)-N-2-(3-cyano-phenoxyethyl)-amine is N-(4′-fluoro-3-biphenylmethyl)-N-2-(3-cyano-phenoxy-ethyl)amine maleate.

Additionally, the invention relates to the use of a pharmaceutical composition for use in veterinary medicine for the treatment of disorders associated with behavioral stressors containing at least a compound being a combined selective dopamine D2 receptor antagonist and 5-HT_{1A} receptor agonist, in particular (R)-(−)-2-[5-(4-fluorophenyl)-3-pyridylmethylaminomethyl]-chromane or one of its physiologically acceptable salts or N(4′-fluoro-3-biphenylmethyl)-N-2-(3-cyano-phenoxy-ethyl)-amine or a physiologically acceptable salt thereof, together with at least one solid, liquid or semiliquid excipient or adjunct for the treatment of disorders associated with behavioral stressors.

Thus the invention provides a pharmaceutical preparation for use in veterinary medicine for the treatment of disorders associated with behavioral stressors characterized in that it contains at least (R)-(−)-2-[5-(4-fluorophenyl)-3-pyridylmethyl-aminomethyl]-chromane hydrochloride and at least one auxiliary substance.

Thus the invention provides a pharmaceutical preparation for the treatment of disorders associated with behavioral stressors for use in veterinary medicine characterized in that it contains at least N-(4′-fluoro-3-biphenylmethyl)-N-2-(3-cyano-phenoxy-ethyl)-amine and/or one of its pharmaceutically acceptable salts and at least one auxiliary substance.

Suitable excipients are organic or inorganic substances which are suitable for enteral (e.g. oral), parenteral or topical adminstration and which do not react with the active compound, for example water, vegetable oils, benzyl alcohols, alkylene glycols, polyethylene glycols, glycerol triacetate, gelatine, carbohydrates such as lactose or starch, magnesium stearate, talc, petroleum jelly. Forms which are used for oral administration are, in particular, tablets, pills, sugar-coated tablets, capsules, powders, granules, syrups, liquids or drops, forms for rectal administration are, in particular suppositories, forms for parenteral administration are, in particular, solvents, preferably oily or aqueous solutions, furthermore suspensions, emulsions or implants, and forms for topical administration are transdermal plasters, ointments, creams or powders. The active compound may also be lyophilized and the resulting lyophilisates used for example for the preparation of injectable products. The abovementioned preparations can be in sterilized form and/or comprise auxiliaries such as glidants, preservatives, stabilizers and/or wetting agents, emulsifiers, salts for modifying the osmotic pressure, buffer substances, colourings, flavourings and/or other active ingredients, e.g. one or more vitamins.

The compounds (R)-(−)-2-[5-(4-fluorophenyl)-3-pyridylmethyl-aminomethyl]chromane and its pharmaceutically acceptable salts or N-(4′-fluoro-3-biphenylmethyl)-N-2-(3-cyano-phenoxy-ethyl)-amine and its pharmaceutically acceptable salts, according to the invention are preferably administered in analogy to other known commercially available preparations for the treatment of disorders associated with behavioral disorders in veterinary medicine. A unit dose will generally contain from 0.1 to 300 mg, preferably between approximately 0.1 (for small birds) and 100 mg (for large dogs), in particular 0.1, 0.5, 1, 5, 10, 30, 50, 100, 200, and 300 mg. The composition may be administered once or more times a day for example 2, 3 or 4 times daily. The daily dose is preferably between approximately 1 and 20 mg/kg of body weight. However, the specific dose for each recepient animal depends not only on the species but on all sorts of factors, for example on the activity of the specific compound employed, on the age, body weight, general state of health, sex, on the diet, on the time and route of administration, on the excretion rate, pharmaceutical substance combination and severity of the particular disorder to which the therapy relates. Oral administration is preferred; the appropriate dosage can typically be mixed with the food without major difficulty, but also peroral routes of administration (e.g. intramuscular or transdermal) can be utilized. To prove the efficacy of the compounds according to the invention, the following clinical studies are described:

### EXAMPLE 1

The aim of the study is to assess the efficacy of the compound treatment of acral lick dermatitis (ALD) in dogs in a multicenter, placebo-controlled study involving veterinarian practitioners (adopted from J. L. Rapoport et al., Arch. Gen. Psychiatr., 1992, 49: 517-521; D. Wynchank and M. Berk, Depress. Anxiety, 1998, 8: 21-23; A. L. Podberscek et al., Vet. Rec., 1999, 145: 365-369).

Forty dogs with ALD are treated with the compound 10 mg/kg once daily, or placebo, for 8 weeks. If necessary, the dose is adjusted according to the clinical response. Owners rate both appearance of the lesion and licking behavior weekly, and veterinarians rate pre- and post-treatment lesions.

### EXAMPLE 2

The aim of the study is to assess the efficacy of the compound treatment of psychogenic alopecia in cats in a multicenter, placebo-controlled study involving veterinerian practitioners (according to K. Seksel and M. J. Lindeman, Aust. Vet. J., 1998, 76: 317-321).

Twenty-eight cats with psychogenic alopecia are treated with the compound with a starting dose of 20 mg/kg once daily, or placebo, for 8 weeks. If necessary, the dose is adjusted according to the clinical response Owners rate both appearance and size of the lesion weekly, and veterinarians rate pre- and post-treatment.

### EXAMPLE 3

The aim of the study is to assess the efficacy of the compound treatment of feather picking in birds in a multicenter, placebo-controlled study involving veterinerian practitioners (according to P. A. Mertens, ESVCE Newsletter, 1997, n^{o} ⅘).

Twentyfour birds with feather picking are treated with the compound 5 mg/kg twice daily, or placebo, for a minimum of 4 weeks and up to 8 weeks. If necessary, the dose is adjusted according to the clinical response. Owners rate both appearance and size of the feather-less lesion, and veterinarians rate pre- and post-treatment (4 weeks and 8 weeks, in case).

1. Use of compounds being combined selective dopamine D2 receptor antagonists and 5-HT_{1A} receptor agonists for the manufacture of a medicament for use in veterinary medicine for the treatment of disorders associated with behavioral stressors.

2. Use according to claim 1 wherein the combined selective dopamine D2 receptor antagonist and 5-HT_{1A} receptor agonist is (R)-(−)-2-[5-(4-fluorophenyl)-3-pyridylmethyl-aminomethyl]-chromane or a pharmaceutically acceptable salt thereof.

3. Use according to claim 2 in which the physiologically acceptable salt is (R)-(−)-2-[5-(4-fluorophenyl)-3-pyridylmethyl-aminomethyl]-chromane hydrochloride.

4. Use according to claim 1 wherein the combined selective dopamine D2 receptor antagonist and 5-HT_{1A} receptor agonist is N-(4′-fluoro-3-biphenylmethyl)-N-2-(3-cyano-phenoxy-ethyl)-amine or a physiologically acceptable salt thereof.

5. Use according to claim 4 in which the physiologically acceptable salt is N-(4′-fluoro-3-biphenylmethyl)-N-2-(3-cyano-phenoxy-ethyl)-amine maleate.

6. Pharmaceutical preparation for use in veterinary medicine for the treatment of disorders associated with behavioral stressors characterized in that it contains at least a combined selective dopamine D2 receptor antagonist and 5-HT_{1A} receptor agonist.

7. Pharmaceutical preparation according to claim 6 characterized in that it contains at least (R)-(−)-2-[5-(4-fluorophenyl)-3-pyridylmethylaminomethyl]-chromane or one of its physiologically acceptable salts and at least one auxiliary substance.

8. Pharmaceutical preparation according to claim 6 characterized in that it contains at least N-(4′-fluoro-3-biphenylmethyl)-N-2-(3-cyano-phenoxyethyl)-amine or a physiologically acceptable salt thereof mane or one of its physiologically acceptable salts.
